(19) United States
(12) Patent Application Publication
(10) Pub. No.: US 2005/0182570 A1
(43) Pub. Date: Aug. 18, 2005

(54) Process for identifying similar 3D substructures onto 3D atomic structures and its applications

(75) Inventors: Christophe Geourjon, Lyon (FR); Martin Jambon, Lyon (FR); Gilbert Deleage, Lyon (FR)

Correspondence Address: IP GROUP OF DLA PIPER RUDNICK GRAY CARY US LLP, 1650 MARKET ST, SUITE 4900, PHILADELPHIA, PA, 19103, US

(73) Assignees: Centre National de la Recherche Scientifique-CNRS an organization of France, Paris Cedex (FR); Universite Claude Bernard Lyon I an organization of France, Villeurbanne Cedex (FR)

(21) Appl. No.: 11/005,346

(22) Filed: Dec. 6, 2004

Related U.S. Application Data
(63) Continuation of application No. PCT/IB03/02928, filed on Jun. 5, 2003.

(30) Foreign Application Priority Data
Jun. 6, 2002 (EP) 02291407.1

(51) Int. Cl. C12Q 1/68; G06F 19/00; G01N 33/48; G01N 33/50

(52) U.S. Cl. 702/19; 702/27

(57) ABSTRACT

The invention pertains to the field of structural biology and relates to a process to compare various three-dimensional (3D) structures and to identify functional similarities among them. The process of comparison of 3D atomic structures of the invention is based on the comparisons of defined chemical groups onto the 3D atomic structures and allows the detection of local similarities even when neither the fold nor sequence for example aminoacid sequences for polypeptides sequences or nucleotide sequences for nucleic acid sequences are conserved. This process requires the attribution of selected physico-chemical parameters to each atom of a 3D atomic structure, then the representation of each 3D atomic structure by a graph of chemical groups.

### RELATED APPLICATION

This is a continuation of International Application No. PCT/IB03/02928, with an international filing date of Jun. 5, 2003 (WO 03/104388, published Dec. 18, 2003), which is based on European Patent Application No. 02291407.1, filed Jun. 6, 2002.

### FIELD OF THE INVENTION

This invention pertains to the field of structural biology and relates to a process to compare various three-dimensional structures and identify functional similarities among them. In particular, the process applies to macromolecules such as proteins.

### BACKGROUND

Understanding and predicting the function of proteins using bioinformatical tools traditionally uses three levels of knowledge: amino acid sequence, backbone fold and local arrangement of atoms. Several tools dealing with sequence or main chain structure are publicly available and routinely used by molecular biologists: tools such as Blast [1] and Fasta [2] provide efficient ways to extract similar sequences from databases containing millions of sequences. There also exist tools that help correlate sequence and function using sequential patterns: the Prosite database [3] consists of human-designed functional signatures that may be searched against a protein sequence.

Profile analysis [4] is a technique based on multiple sequence alignments of homologous sequences and may be used to test a sequence for its membership in a family. Pattinprot [5] facilitates searching a database for any given pattern that may have been inferred from multiple sequence alignments such as those obtained with ClustalW [6] from a set of homologous protein sequences. When a 3D structure of a given protein is available, it is possible to use tools such as the Dali/FSSP server [7, 8] that mainly use the main chain to find similarities and classify proteins. However, these process reach their limits in many cases: a significant similarity in the sequence or in the fold of two proteins is neither necessary nor sufficient to prove that they share a common biological function.

Inferring biological function from 3D structures of proteins is still a difficult problem, given that it strongly depends upon the biological context surrounding every protein molecule in vivo. However, precisely analyzing data provided by crystallographic or NMR experimental studies may show local structural similarities across various proteins that could be correlated to an already known biological function. Although significant efforts have been spent over the past years on developing surface matching algorithms, very few methods combine chemical information together with geometry in an efficient manner, and none of them use custom chemical groups as the elementary bricks responsible for biochemical activity.

Methodologies based on computer vision heuristics have been developed in the 90's [9]. These processes are purely geometrical and use discretized representations of the molecular surface. Variants and improvements of the original technique select sparse critical points among all points representing the molecular surface [10] and introduce a small number of hinges allowing flexibility in the docking or matching process [11]. Other tools use the surface representation of the proteins to perform comparisons by other means [12-14]. The chemical environment has been successfully taken into account in predictive studies concerning special cases: metal-binding sites [15, 16] and sugar-binding sites [17]. Thus, the challenge was to provide a generic tool that returns satisfying results for a large number of protein functions without manual or statistical tuning.

Several methods implementing comparison of the protein sequences or folding have been already developed. Nevertheless, most of them are only able to reflect similarities found at the surface of the 3D objects.

### SUMMARY OF THE INVENTION

This invention relates to a process for identifying similar 3D substructures onto 3D atomic structures having a plurality of individual atoms including the steps of a) attributing to each individual atom of the 3D atomic structure a structural parameter combining atomic local density D, local center of mass C and orientation in relation with position P; b) constructing chemical groups by setting individual atoms having similar structural parameters; c) constructing clusters of at least three chemical groups by setting the chemical groups whose reciprocal distances are constrained; and d) comparing clusters constructed in step (c) and identifying the clusters sharing similar 3D structures.

### BRIEF DESCRIPTION OF THE DRAWINGS

The invention will be illustrated hereafter with some obtained experimental results and in view of FIGS. 1 to 12 wherein:

FIGS. 1 to 7 illustrate major steps of the process of comparison of 3D structures according to aspects of the invention.

FIG. 1 shows the local density computation around a given atom A with a plot representing the weight function.

FIG. 2 illustrates an example of two chemical groups as currently defined.

FIG. 3 represents the different vectors useful to represent geometric information associated to each chemical group and related to their spatial orientation.

FIG. 4 illustrates reduction of atoms to chemical groups (A), followed by selection of chemical groups according to the user's will (B). This figure reflects the particular embodiment in which three chemical groups are selected to form triangles graphs (C), followed by a computation of parameters associated to each triangle (D). Letters are those used in the text: P₁, P₂ and P₃ represent the position of the chemical groups, C₁, C₂ and C₃ represent the local centers of mass associated to the chemical groups, P and C are the centers of these points.

FIG. 5 corresponds to an input of graphs from a database (A) and illustrates the main comparison step (B).

FIG. 6 illustrates an optional refinement step.

FIG. 7 illustrates the process of obtaining families of substructures from an arbitrary number of structures, by starting using pairwise comparison results. The example shows 3 molecules denoted by M1, M2 and M3 as in the text.

FIGS. 9 to 11 illustrate the screening results and information about the legume lectin family.

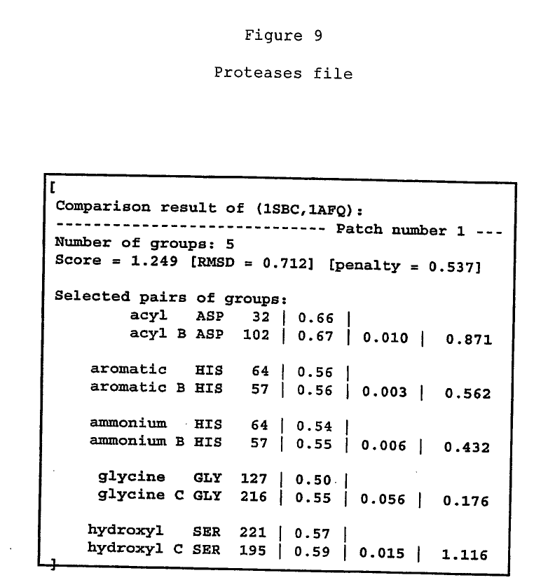

FIGS. 8 and 9 illustrate the comparison of serine proteases: subtilisin, 1SBC structure vs. chymotrypsin, 1AFQ structure.

FIG. 8 is a schematic view of the sequences of both functional forms of the serine proteases with highlighting of the catalytic triad residues.

FIG. 9 shows the computer file result obtained by applying the process of the invention to proteases 3D structures.

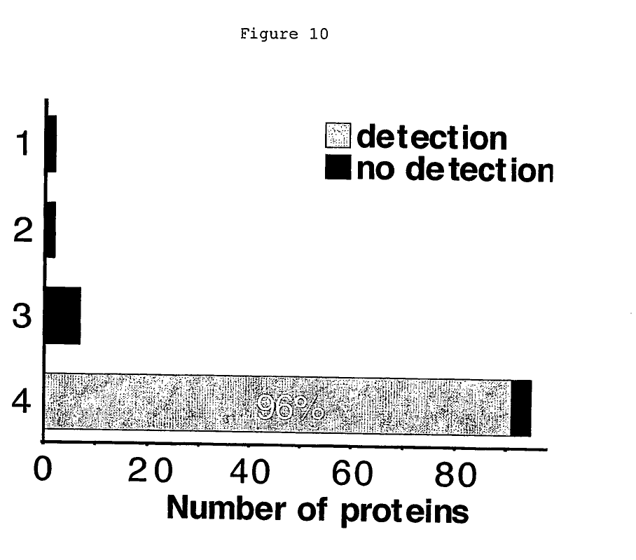

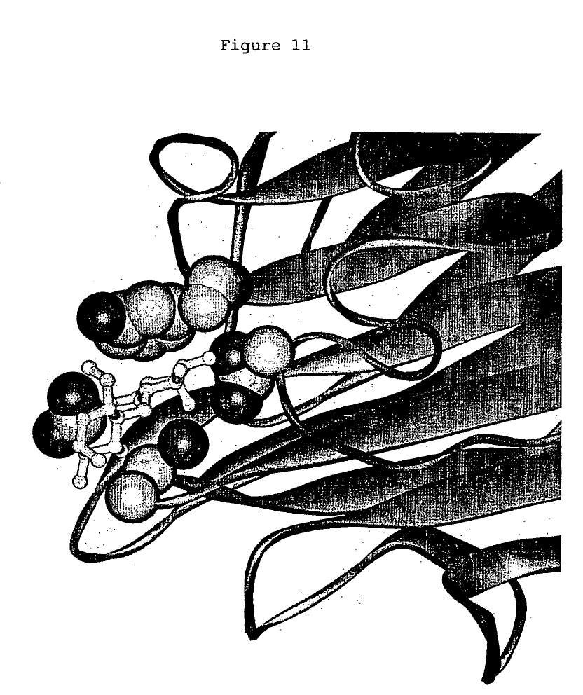

FIG. 10 illustrates results obtained by the process of comparison of the invention; no incorrect patch was returned.

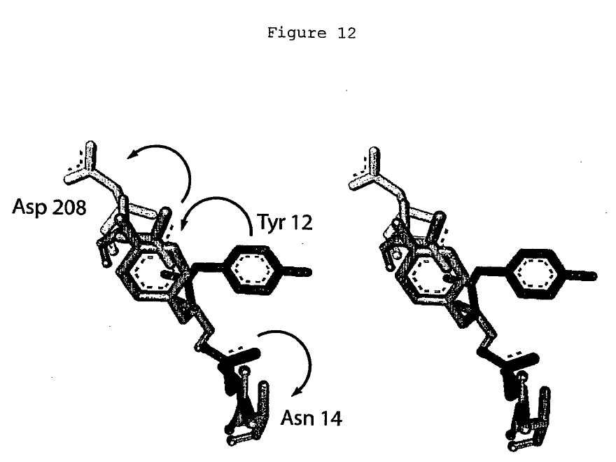

FIG. 11 is a view of the chemical groups defining the sugar-binding site in the peanut lectin structure 2PEL.

FIG. 12 illustrates essential aminoacids for sugar binding in concanavalin A before and after demetallization; superposition of α-carbons with 0.9 Å RMSD of 1DQ1 (native form) and 1DQ2 (apoprotein).

### DETAILED DESCRIPTION

We have now developed a new process of identifying similarities between the 3D atomic structures, even if those similarities are not exposed over the surface of the 3D atomic structures. The process we developed is not based, as most of previous ones, on comparison of linear sequences nor comparison of folding structures.

The process of comparison of 3D atomic structures of the invention is based on comparisons of defined chemical groups onto the 3D atomic structures and allows detection of local similarities even when neither the fold nor the sequence, for example amino acid sequences for polypeptides sequences or nucleotide sequences for nucleic acid sequences, are conserved. This process uses attribution of selected physico-chemical parameters to each atom of a 3D atomic structure, then the representation of each 3D atomic structure by a graph of chemical groups, i.e. if the chemical groups are selected by forming triplets, then the graph is represented by triangles.

Then, the starting point of the representation of 3D atomic structures is the definition of chemical groups within the structure. Some atoms may not belong to any of the chemical groups, whereas some others may be part of several groups as illustrated in Table 1.

Table 1 illustrates the definition of some chemical groups that may be used in the process of the invention, although other definitions can be specified by the user.

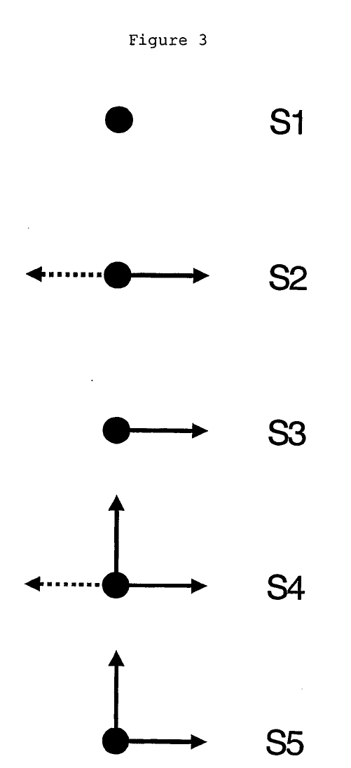

The chemical group description groups done in Table 1 show an example of correspondence between chemical groups and amino acids. Column 3 shows amino acids that contain at least one of the given chemical group from Column 1. Column 4 indicates the geometric construction that is associated to the given chemical group, as defined in FIG. 3.

TABLE 1

| Chemical group | | | |
|---|---|---|---|
| Description | Symbol | Amino acids | Geometry |
| Carboxylic acid | acyl | Asp, Glu | S4 |
| Primary amide | amide | Asn, Gln | S5 |
| Aromatic ring | aromatic | His, Phe, Trp, Tyr | S2 |
| Free δ⁺ hydrogen | delta_minus | all | S3 |
| Free δ⁻ atom | delta_plus | all except Pro | S3 |
| Glycine | glycine | Gly | S3 |
| Guanidinium group | guanidinium | Arg | S2 |
| Imidazole group | histidine | His | S5 |
| Hydroxyl group | hydroxyl | Ser, Thr, Tyr | S3 |
| Proline | proline | Pro | S1 |
| Methionine sulphur | thioether | Met | S1 |
| Cysteine thiol | thiol | Cys | S3 |

The 3D atomic structures are preformatted before any comparison. This operation usually takes longer than the comparison itself. Thus, the preformatted data may be stored in a database to be reused later.

1. Chemical Groups Construction

Chemical groups are defined as sets of atoms that share strong geometric constraints and in a way that focuses on their potential interaction with target molecules. Hydrogen bond donors, hydrogen bond acceptors and aromatic rings are examples of potential interactors with target molecules and, thus, may constitute chemical groups within the meaning of the present invention.

For purposes of this invention, chemical groups are 3D atomic substructures having a common set of physico-chemical parameters including:
I. Spatial position where the function of the chemical group takes place, refered as functional position or position,
II. Spatial position of their center of mass, refered as physical position,
III. Group-specific information.

Additional geometric information is associated to each chemical group. The form of this information is specific to each kind of chemical group, since it is only required for the comparison and scoring of chemical groups of the same kind.

The following geometric objects can be used to represent this group-specific information (FIG. 3):

S1: empty information. This can be used to represent isotropic objects such as a charge.

S2: non oriented symmetry axis. This can be used to reflect symmetric bipolar objects such as aromatic rings.

S3: simple polarisation. This represents an orientation in a single direction. This representation may be useful to represent hydrogen bond donors and acceptors.

S4: semi-symmetric double polarisation. This is an oriented object like S3 in which the perfect symmetry around the axis is replaced with a 2-order symmetry around the axis. S4 could be used to represent carboxylic groups in their basic form.

S5: double polarisation. S5 may be used to represent objects with no symmetry axis such as amide groups.

Geometrical contructs S1, S2, S3, S4 and S5 are defined using vectors in addition to the spatial position of the chemical groups, as illustrated in FIG. 3.

Chemical groups according to aspects of the invention are independent from comparison algorithms and, thus, may be changed according to the user's requirements/desires.

1.1 Local Density

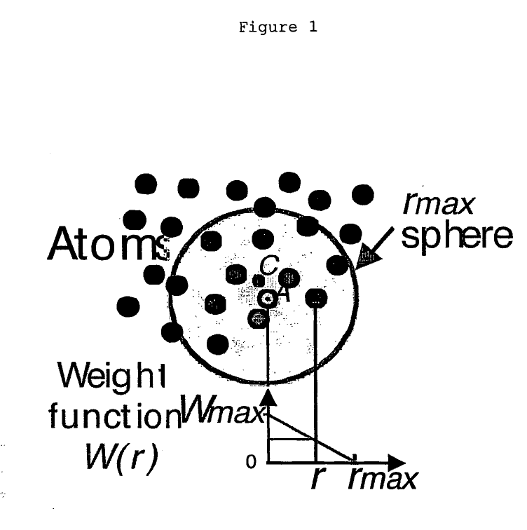

The parameter called “local density” D is calculated for each atom A occupying a spatial position P (FIG. 1) and used in the comparison process. Its purpose is to give a discriminative estimation of the burial of an atom.

The burial of atoms may be estimated using a continuous local atomic density function. The general expression of a local density D(xₚ, yₚ, zₚ) around the position P is:

$$D ( {x}_{P} , {y}_{P} , {z}_{P} ) = \frac{{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot m ( x , y , z ) \cdot ⅆ x \cdot ⅆ y \cdot ⅆ z}{{\int }_{- ∞}^{+ ∞} {\int }_{- ∞}^{+ ∞} {\int }_{- ∞}^{+ ∞} w ( x , y , z ) \cdot ⅆ x \cdot ⅆ y \cdot ⅆ z}$$

where x, y and z are spatial coordinates, m is the density function and w is a weight function to reduce the influence of the peripheral atoms around a given one.

A spherical weight function can be used:

$$w ( x , y , z ) = \frac{1}{4} ( 1 - \frac{r}{{r}_{C}} )$$

if r≦r_{c}, 0 otherwise
where r is \sqrt{x²+y²+z²} and r_{c} a critical radius. Factor [¼] allows to make r independent from r_{c} if m is constant.

Burial of a given chemical group may then be estimated by two alternative means:
a) calculating the arithmetic mean of the local atomic densities around each atom belonging to this group;
b) by using the local atomic density for the position of its center.
   1.2 Local Center of Mass.

For each atom with center P, a vector that indicates the exterior of the 3D atomic structure is computed.

This vector indicating the exterior of the 3D atomic structure may be represented by a density gradient.

Vector \vec{CP} wherein point C is the local center of mass of atom A occupying a position P may be used.

The local center of mass C(P) for point P is a point which cartesian coordinates (x_{c}, y_{c}, z_{c}) match the following formulation:

$$\{ \begin{matrix}{x}_{C} ( {x}_{P} , {y}_{P} , {z}_{P} ) = \frac{{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot m ( x , y , z ) \cdot x \cdot ⅆ x \cdot ⅆ y \cdot ⅆ z}{{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot m ( x , y , z ) \cdot ⅆ x \cdot ⅆ y \cdot ⅆ z} \\ {y}_{C} ( {x}_{P} , {y}_{P} , {z}_{P} ) = \frac{{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot m ( x , y , z ) \cdot y \cdot ⅆ x \cdot ⅆ y \cdot ⅆ z}{{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot m ( x , y , z ) \cdot ⅆ x \cdot ⅆ y \cdot ⅆ z} \\ {z}_{C} ( {x}_{P} , {y}_{P} , {z}_{P} ) = \frac{{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot m ( x , y , z ) \cdot z \cdot ⅆ x \cdot ⅆ y \cdot ⅆ z}{{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot m ( x , y , z ) \cdot ⅆ x \cdot ⅆ y \cdot ⅆ z}\end{matrix}$$

where x, y and z are spatial coordinates, m is the density function and w is a weight function.

The weight function w may be defined similarly to the weight function used in the local density expression defined in section 1.1.

1.3 Orientation

Orientation of each atom A occupying the position P and associated to the local center of mass C, is performed by the vector \vec{CP} that points toward the exterior of the 3D atomic structure. The notion of exterior depends on the weight function that has been adopted for the calculation of the center of mass.

For all kinds of molecules, using a weight function w explicitly defined in section 1.1, a critical radius r_{c} ranging from 3 to 50 Å, preferably from 5 to 20 Å is used.

Once defined and calculated, physico-chemical parameters that characterize the environment surrounding each atom are computed.

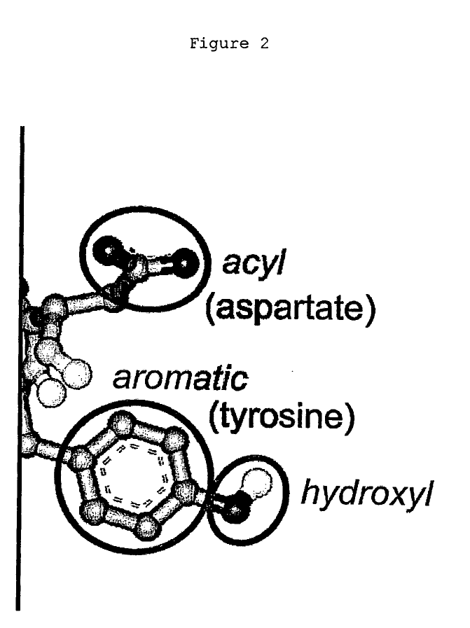

Every chemical group of the 3D atomic structure comprises a set of atoms as defined in the input file (FIG. 2). Thus, for a given chemical group, the mean position P of these atoms, the mean position C of the local centers of mass and the mean local density D are computed and recorded.

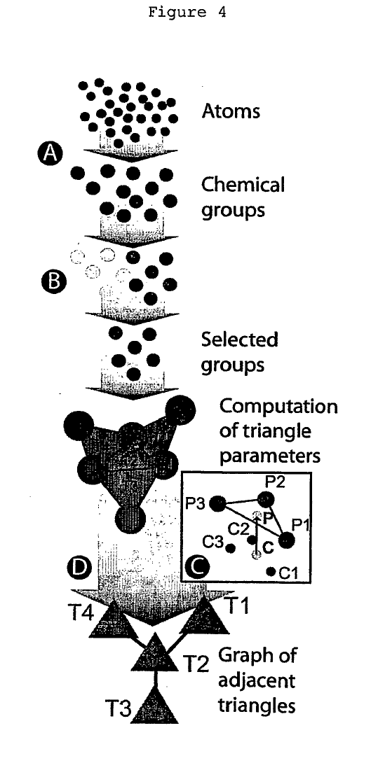

This step reduces the representation of the 3D atomic structure by a set of chemical groups instead of atoms (FIG. 4A).

1.4 Further Selection of Chemical Groups

At this stage, a selection over the chemical groups is performed (FIG. 4B). The following procedures allow the user to select specific parts of the molecular structure:
a) automatic selection of most exposed chemical groups using a local density function and a threshold,
b) semi-automatic selection of chemical groups that are possibly interacting with a given set of chemical groups,
c) manual selection of subsets of chemical groups.

Step (b) is based on the definition for each chemical group of a set of points called virtual interactors.

A chemical group in a given molecule is denoted (P,L) where P is its position and L is the set of points constituting the virtual interactors. A given group (Pᵢ,Lᵢ) is said to be interacting with (P,L) if and only if there exists at least one point Q belonging to Lᵢ such that PQ≦dₘₐₓ, where dₘₐₓ is an empirical threshold.

For example, two virtual interactors can be defined for aromatic rings, each being located symmetrically on both sides of the aromatic ring at a distance of 4 Å of its center.

2. Cluster Construction (3D Substructures).

Once constructed, sets of chemical groups, constituting 3D substructures, are selected to construct clusters of at least three chemical groups by setting the chemical groups whose reciprocal distances are positioned inside a sphere of radius r, where the radius is between 2 to 20 Å, preferably between 2 to 8 Å.

An algorithm for finding neighboring points in constant time is used, so that the complexity of this step is proportional to the global number of chemical groups, given a definitive cluster size.

In a particular aspect of the process to identify 3D substructures, the sets of chemical groups are selected in such a way that they comprise three chemical groups (triplets). Then, the sets of chemical groups that represent the 3D atomic structure are converted to triangles of chemical groups. In this particular aspect, each triplet (A, B, C) of a chemical group is rejected if the distance between the physical position of two groups among A, B, and C is higher or lower than given distance thresholds.

Sets of chemical groups are oriented against the surface, for instance, in a particular example of sets of three chemical groups, the orientation of a triangle (P1; P2; P3) of chemical groups is estimated, for example, by using a scalar triple product of \vec{CP}₁, \vec{CP}₂ and \vec{CP}₃, wherein C is the center of the local centers of mass of P1, P2 and P3.

Once the triangles are obtained, the set of triangles representing one 3D atomic structure is converted into a graph
in which
   each vertex represents one triangle;
   each edge represent the adjacency of two triangles.

Also, additional parameters may be added to this graph. For instance, the angle between the adjacent triangles is associated to each edge in the graph.

For every triangle (FIG. 4C) formed by three chemical groups (P1, P2, P3) with edges shorter than a given threshold several parameters are computed:
(1) distances between two vertices of a triad are computed and recorded;
(2) the burial of each chemical group is estimated using the local density;
(3) the orientation of the triangle towards the rest of the 3D atomic structure is estimated by the scalar triple product of (\vec{CP}₁, \vec{CP}₂, \vec{CP}₃), C being the local center of mass of the triad, i.e. the center of C₁, C₂ and C₃ which are the local centers of mass of the chemical groups located at P₁, P₂ and P₃. The final representation of the 3D atomic structure is obtained by connecting adjacent triangles, i.e. triangles that share exactly two chemical groups, to make a graph in which each triangle forms a vertex (FIG. 4D).
   3. Pairwise Comparison of the 3D Atomic Structures.

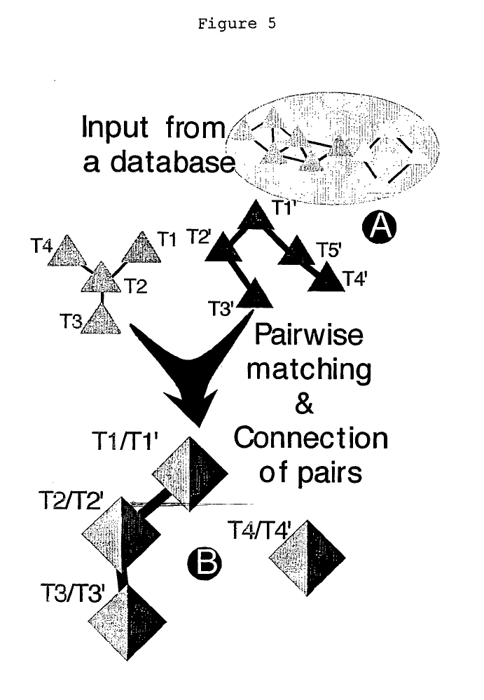

Once the 3D atomic structures to be compared are selected, and once the construction of clusters of chemical groups, i.e. triangles comprising triplets of chemical groups onto each one of the 3D atomic structures is completed, a comparison of the structures can be generated or may be retrieved from a database (FIG. 5A).

Then, the process of comparing two 3D atomic structures comprises the steps hereafter described:

3.1 Searching of Pairs of Similar Clusters of Chemical Groups. (i.e. Triplets)

The criteria for pair similarity are various and can be selected among the group consisting of:
(a) same kind of chemical groups,
(b) similar orientation of the equivalent chemical groups in the two triangles once the triangles have been superposed, after a scoring function specific to each kind of chemical group,
(c) the same length of equivalent edges in the two triangles,
(d) the similar burial of the equivalent chemical groups in the two triangles (local density),
(e) the similar orientation of the two triangles, regarding the rest of the 3D atomic structure (scalar triple product).
   3.2 The Calculation of a Score Indicating the Level of Similarity of Clusters of Said Pair.

The score is then computed.

The criteria for selecting a given pair of clusters are the following:
a) every score associated with a given parameter must be above a given threshold; the threshold being either constant or dependent on the type of chemical group or the environment. A parameter has been designated to compare each chemical group, for example, aromatic group and guanidium group correspond both to a <<bipolar>> construction, but the first one has an <<angle>> parameter with a value of 60 degrees and the second one has an <<angle>> parameter with a value of 45 degrees.
b) he global score for the pair of clusters must be above a given threshold. The global score combines individual scores obtained at step (a), by using a linear combination of these scores.

Thresholds for individual scores at step (a) or global score at step (b) may be also designed empirically, possibly using automated optimizations based on statistical studies.

In the particular embodiment of clusters of three chemical groups, once selected, each pair of similar triplets forms a vertex in a comparison graph.

Given two graphs of triangles, representing two 3D atomic structures M₁ and M₂, vertices T_{1,1} and T_{1,2} in 3D atomic structure M₁, and vertices T_{2,1} and T_{2,2} in the 3D atomic structure M₂, the criteria for connecting pairs (T_{1,1}; T_{2,1}) and (T_{1,2}; T_{2,2}) in the comparison graph are the following:
(a) T_{1,1} and T_{1,2} must be connected (adjacent) in M₁;
(b) T_{2,1} and T_{2,2} must be connected (adjacent) in M₂;
(c) the angle between T_{1,1} and T_{1,2} must be similar to the angle between T_{2,1} and T_{2,2}.

The independent subgraphs in the comparison graph for the 3D atomic structures M₁ and M₂ represent then a set of pairs of equivalent triangles corresponding to two structurally equivalent regions in 3D atomic structures M₁ and M₂.

3.3 Optimization Steps

To optimize the obtained results, the process of the invention could further comprise one or several additional steps.

The first results conducted to sets of pairs of similar triangles. The pairs of similar triangles are converted into pairs of chemical groups. Such sets of pairs of converted chemical groups are sometimes hereinafter called “patches.”

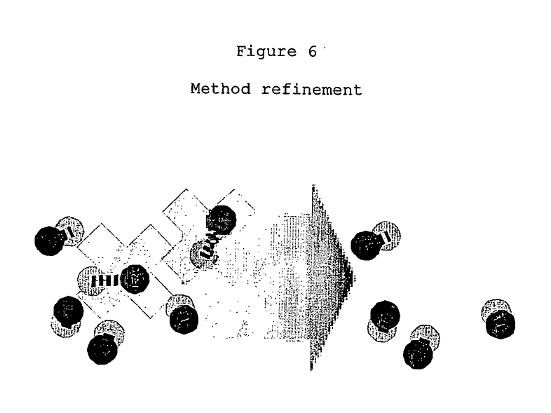

The patches are then refined using a selection procedure (FIG. 6) comprising the following steps:

1) Pairs of chemical groups within a patch may be superposed by minimizing a distance function. The distance function is the following:
dist(g₁,g₂)=α·∥pos(g₁)−pos(g₂)∥+β·|D(g₁)−D(g₂)|+γorient(g₁,g₂)
where pos(g) is the position of the chemical group g after optimal superimposition of the given set of pairs, and D(g) its local density; _{orient(g}_{₁}_{,g}_{₂}₎ is the difference of orientation between chemical groups g₁ and g₂ after optimal superimposition. α, β and γ are weighting coefficients that are defined on an empirical basis.

2) Several cycles with different elimination thresholds may be performed successively or in parallel by
a) calculating a score including superposition quality and the number of pairs in the patch and
b) elimination of patches having a score inferior to the current threshold.

3) Iterative steps comprising both steps of calculation and elimination may be performed to lead to a final score.

4) Finally, patches whose final score is below a given threshold are not retained.

At this step, the definitive patches are known among others. An additional parameter called atomic volume difference is computed for each patch and is used as an additional criterion for:
a) eliminating patches with low shape similarity,
b) building a more relevant score.

The purpose of the computation of the atomic volume difference is to compare the volumes of three kinds of atoms for a given patch:
V₁: atoms surrounding chemical groups in cluster (3D atomic substructure) extracted from M₁;
V₂: atoms surrounding chemical groups in cluster (3D atomic substructure) extracted from M₂;
V: atoms surrounding chemical groups in both clusters after superposition of the said clusters.

If V₁ and V₂ are similar, and if V is similar to V₁ and to V₂, then the repartition of the atoms around the selected groups is similar.

If V₁ and V₂ are similar, but V is much higher than V₁ or V₂, then the repartition of the atoms around the selected chemical groups is much different.

A score that represents atomic volume difference is derived from these calculations.

Here is an example of such a function:

$$s ( {v}_{1} , {v}_{2} , v ) = 1 - \frac{2 v - ( {v}_{1} + {v}_{2} )}{v}$$

In a particular example, the 3D substructures of the 3D atomic structures are compared to obtain a list of similar 3D substructures that are associated to a score that combines several of the following criteria on an empirical basis:
a) deviation after optimal superimposition,
b) average difference of local density between chemical groups,
c) difference in the orientation of the superimposed chemical groups, using a specific scoring function for each kind of chemical group,
d) volume of the chemical groups,
e) difference in the shape of the patches by means of an atomic volume difference calculation.
   4. Multiple Comparison of 3D Atomic Structures.
   4.1 Introduction

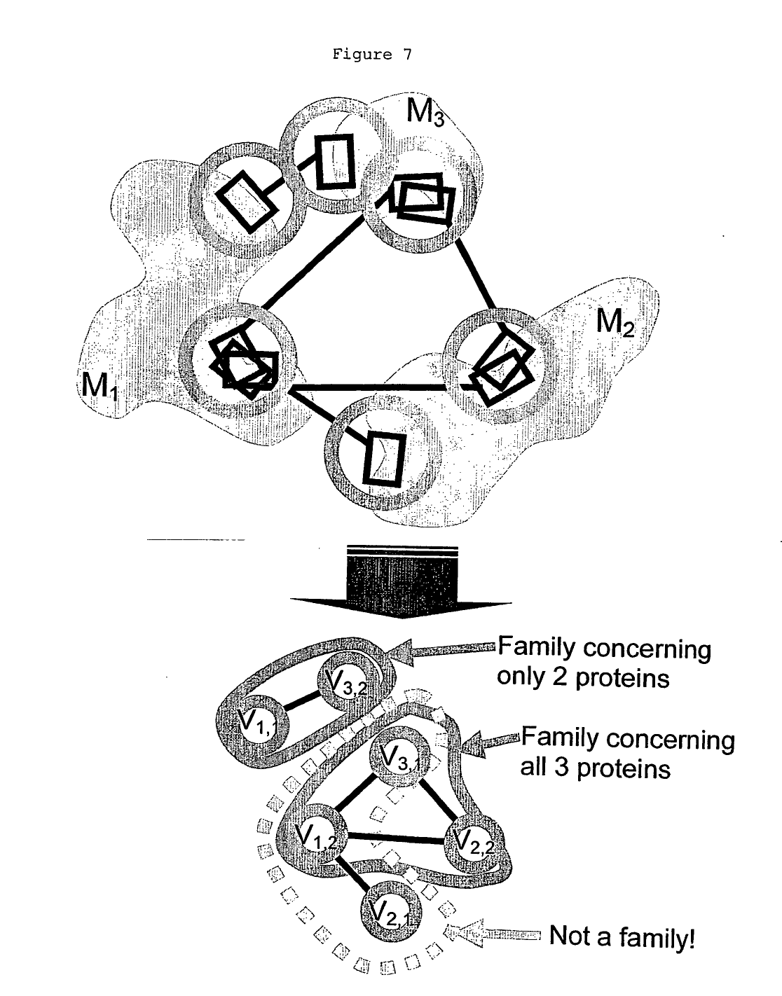

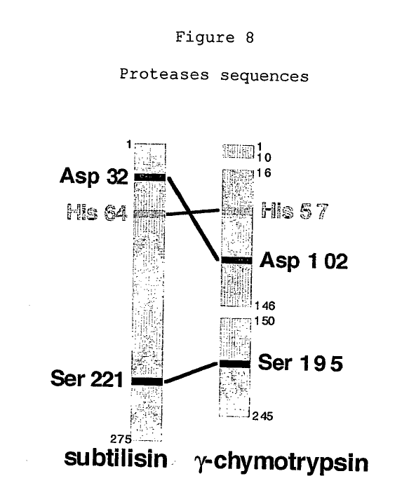

The process for finding similarities across several molecular structures relies on the pairwise comparison of all structures. This process is schematically illustrated on FIG. 7. The result is a set of families that have the following properties:
a) a given family does not necessarily concern all molecules that are being compared. Thus, this process is not influenced by the addition of a foreign molecular structure that does not share any similarity with the other molecular structures;
b) a family is a set of elements that stand for a set of chemical groups belonging to a single molecular structure;
c) each chemical group belonging to a given member of a family can be associated to a coefficient that indicates its frequency within the family, and therefore its importance in this family.
   4.2 Preliminary Definitions
   Definition 1

A “clique” is a complete subgraph from a given graph.

Definition 2

For a real parameter λ in the range [0;1], a λ-clique C in a given graph G is a subgraph such that every vertex from C is connected to at least λ·(|C|−1) vertices from C, where |C| is the cardinality of C.

Thus, clique is a synonym for 1-clique.

Definition 3

A “maximal clique” is a clique which is not a subset of any other clique.

Definition 4

A “maximal λ-clique” is a λ-clique which is not a subset of any other λ-clique.

4.3 Description of the Process

4.3.1 Clusterization of Overlapping Sets of Chemical Groups

Let M be the set of molecular structures:
M={M₁, . . . ,Mₙ}

For a given structure Mᵢ comprising a set of chemical groups:

Mᵢ={g_{i,1}, . . . ,g_{i,|Mi|}}

The applicant defines a comparison function that returns a set of sets of pairs(P) of chemical groups:
comparison(Mᵢ,Mⱼ)={P_{i,j,1},P_{i,j,2,}, . . . ,P_{i,j,|conparison(M}_{ᵢ}_{,M}_{_{j)|}}}
where P_{i,j,k}={(g_{i,αk,1},g_{j,βk,1}),(g_{i,αk,2},g_{j,βk,2}), . . . ,(g_{i,αk,|Pi,j,k|},g_{j,βk,|Pi,j,k|})}.

Extraction of the chemical groups belonging to each of the structures is then operated by means of the following functions s₁ and s₂:

$$\vert \begin{matrix}{s}_{1} ( {P}_{i , j , k} ) = \{ {g}_{i , {α}_{k , 1}} , {g}_{i , {α}_{k , 2}} , … , {g}_{i , {α}_{k , \vert {P}_{i , j , k} \vert}} \} \\ {s}_{2} ( {P}_{i , j , k} ) = \{ {g}_{j , {β}_{k , 1}} , {g}_{j , {β}_{k , 2}} , … , {g}_{j , {β}_{k , \vert {P}_{i , j , k} \vert}} \}\end{matrix}$$

Then Pᵢ is defined as follows:
Pᵢ={s₁(P_{i,j,k})|1≦j≦m and P_{i,j,k} εcomparison(Mᵢ,Mⱼ)}.

Then, for a given molecule Mᵢ, the method comprises the construction of a graph Gᵢ including:
a) vertices that match the elements of Pᵢ,
b) edges that connect any vertices u and v sufficiently overlapping according to a given predicate such as the following:

$$\frac{\vert u ⋂ v \vert}{max ( \vert u \vert , \vert v \vert )} \geq overlap$$

In this particular aspect, overlap is a real number within the range 0 and 1,preferably between 0,5 and 1 and more preferably 0,7.

The maximal cliques from Gᵢ are denoted as follows:
maximal-cliques(Gᵢ)={V_{i,1},V_{i,2}, . . . ,V_{i,vi}}
   4.3.2 Extraction of Families

To operate the extraction of the families of similar clusters of chemical groups that are found similar in several molecular structures, the following function is defined:
families(M)=maximal-λ-cliques(H(M))
wherein H(M) is the graph such that:

$$a ) \qquad vertices = {⋃}_{i = 1}^{m} maximal - cliques ( {P}_{i} )$$

b) there is an edge connecting vertices V_{i,a} and V_{j,b} if and only if:

$$∃ k such that \{ \frac{{s}_{1} ( {P}_{i , j , k} ) ∈ {V}_{i , a}}{{s}_{2} ( {P}_{i , j , k} ) ∈ {V}_{j , b}} .$$

Note that a cluster V of chemical groups is a set of overlapping sets of chemical groups. V can be converted into a set {(g₁,w₁),(g₂,w₂), . . . } where each chemical group gᵢ is associated to a weight wᵢ that may range from 1 to |V| and is the number of occurrences of this chemical group in the elements of V.

4.4 Algorithms

The problem of finding the maximum clique in an arbitrary graph, i.e. the maximal clique of maximum cardinality, is known to be NP-complete. Therefore, the problem of getting all maximal cliques and the larger problem of getting all maximal □-cliques are also NP-complete.

However, this has not be proven for the particular cases that are considered here. The algorithm that is used for finding all maximal λ-cliques is a clustering algorithm: subgraphs are initially constituted of individual vertices and are extended using neighbor vertices under the condition that the resulting subgraph is still a λ-clique. Redundant subgraphs are removed during the process.

The applicant found that the value of 0.7 is a suitable value for both overlap and parameters in the multiple comparison process.

5. Application: Database Design-Screening

5.1 Design of Databases for Efficient Screening

Database containing 3D atomic substructures of 3D structures may be preformatted to allow a quick comparison of one of its members to any other 3D structure preformatted.

The design of the databases can be applied to any kind of 3D atomic structures such as proteins, nucleic acids or other natural and artificial polymers, but also non-polymeric atomic 3D structures.

The database may comprise complete 3D atomic substructures, but also fragments thereof.

In particular, the database may comprise particular fragments of 3D atomic structures implicated in biological processes, such as enzymatic processes, reversible or irreversible binding of a class of molecules, sensitivity to a particular physico-chemical environment, energy conversion, self modification, antigenicity, modification of the intensity of a biological process.

The fragments of 3D atomic substructures to be included in the database may also be determined automatically, by selection of chemical groups that interact with a ligand which is present in the 3D atomic structure or predicted by biochemical experiences.

As a ligand one may understand a 3D atomic structure, whatever is its nature, such as a peptide or an oligonucleotide, able to bind to another molecular partner, as a receptor, an antibody, a co-factor.

Selection of the sites of interaction between the 3D atomic structure and the ligand may be made by characterizing positioning around each chemical group included in essential regions of the 3D atomic structure.

In a particular aspect, each chemical group is associated with a set of target positions. A target position is a spatial position with high probability for finding a molecular interactor. The number of target positions for a given chemical group may depend on its chemical environment.

More precisely, the invention relates to a process to identify similar 3D substructures onto 3D atomic structures having a plurality of individual atoms, performed with the aid of a programmed computer comprising:
a) attributing to each individual atom of the 3D atomic structure a structural parameter combining its atomic local density D, its local center of mass C and its orientation in relation with its position P,
b) constructing chemical groups by setting individual atoms having similar structural parameters,
c) constructing clusters of at least three chemical groups by setting the chemical groups whose reciprocal distances are constrained, and
d) comparing clusters constructed at step (c) and identifying the clusters sharing similar 3D structures.

The atomic local density D of step (a) may be calculated for each atom A, on basis to its spatial position P defined by its coordinates (xₚ, yₚ, zₚ) as a function of its density m, modulated by a weight function w, by means of the function:

$$D ( {x}_{P} , {y}_{P} , {z}_{P} ) = \frac{{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot m ( x , y , z ) \cdot ⅆ x \cdot ⅆ y \cdot ⅆ z}{{\int }_{- ∞}^{+ ∞} {\int }_{- ∞}^{+ ∞} {\int }_{- ∞}^{+ ∞} w ( x , y , z ) \cdot ⅆ x \cdot ⅆ y \cdot ⅆ z}$$

And the weight function w is preferably calculated as a spherical function

$$w ( x , y , z ) = \frac{1}{4} ( 1 - \frac{r}{{r}_{C}} )$$

if r≦r_{c}, 0 otherwise
wherein r is \sqrt{x²+y²+z²}, r_{c} a critical radius and the factor [¼] allows to make r independent from r_{c} if m is constant.

The local center of mass C(P), for a given atom A occupying a position P which Cartesian coordinates (x_{c}, y_{c}, z_{c}) preferably match the following formulation:

$$\{ \begin{matrix}{x}_{C} ( {x}_{P} , {y}_{P} , {z}_{P} ) = \frac{ \begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot x \cdot ⅆ x \cdot ⅆ y \cdot ⅆ z\end{matrix}}{\begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot ⅆ x \cdot ⅆ y \cdot ⅆ z\end{matrix}} \\ {y}_{C} ( {x}_{P} , {y}_{P} , {z}_{P} ) = \frac{ \begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot y \cdot ⅆ x \cdot ⅆ y \cdot ⅆ z\end{matrix}}{\begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot ⅆ x \cdot ⅆ y \cdot ⅆ z\end{matrix}} \\ {z}_{C} ( {x}_{P} , {y}_{P} , {z}_{P} ) = \frac{ \begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot z \cdot ⅆ x \cdot ⅆ y \cdot ⅆ z\end{matrix}}{\begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot ⅆ x \cdot ⅆ y \cdot ⅆ z\end{matrix}}\end{matrix}$$

wherein x, y and z are spatial coordinates, m is the density function and w is a weight function.

In a particular aspect of the process, the weight function w is a spherical function:

$$w ( x , y , z ) = \frac{1}{4} ( 1 - \frac{r}{{r}_{c}} )$$

if r≦r_{c}, 0 otherwise
wherein r is \sqrt{x²+y²+z²}, r_{c} a critical radius and the factor [¼] allows to make r independent from r_{c} if m is constant.

Also, orientation of each atom A occupying a position P and having a local center of mass C(P) at step (a) of the process, may be preferably calculated by means of a density gradient represented by a vector \vec{CP}.

In a preferred aspect of this process to identify similar 3D substructures, the reciprocal distances between chemical groups selected in step (c) are 2 to 20 Å, preferably 5 to 12Å.

Also, construction of clusters at step (c) comprises orientation of the clusters against the 3D atomic structure and preferably this orientation is operated by a scalar triple product of three vectors \vec{CP}ᵢ, \vec{CP}ⱼ, \vec{CP}ₖ, wherein C is the center of the local centers of mass of each chemical group and Pᵢ, Pⱼ, Pₖ are three distinct points in the cluster.

The process to identify similar 3D substructures is particularly useful to construct clusters of chemical groups that can be further stored and classified in a database.

Comparison of a given pair of clusters at step (d) of a such process to identify similar 3D substructures comprises the identification of at least one structural similarity selected from the group consisting of:
(a) same chemical groups and similar orientation,
(b) similar length of reciprocal distances between chemical groups,
(c) similar local density of the chemical groups,
(d) similar orientation of the constructed clusters, and
(e) capability of binding flexible ligands using the same kind of weak chemical bonds.

Furthermore, after identification of at least two structural similarities, the process comprises calculation of a global score, that may be calculated as a function combining several parameters indicating the similarity of the clusters, the parameters being selected among the group consisting of:
volume of chemical groups,
scarcity of the chemical groups,
quality of the superposition with respect to the standard deviation,
quality of the superposition with respect to the orientation of the chemical groups, and
resemblance of the atomic environment.

The process may be applied to the capability of binding flexible ligands using the same kind of weak chemical bonds. In this way, the capability may be estimated by analysis of deviation of short range distances between chemical groups.

In a particular aspect, the resemblance between atomic environment comprises calculation of a score by comparing volumes of atoms around each converted pair of chemical groups and a such score is calculated by the function:

$$s ( {v}_{1} , {v}_{2} , v ) = 1 - \frac{2 v - ( {v}_{1} + {v}_{2} )}{v}$$

wherein:
V₁ is the volume of atoms surrounding chemical groups in a cluster (3D atomic substructure) extracted from atomic structure M₁;
V₂ is the volume of atoms surrounding chemical groups in a cluster (3D atomic substructure) extracted from atomic structure M₂; V is the volume of atoms surrounding chemical groups in both clusters after superposition of the clusters.

Also, in a particular aspect, the process to identify similar 3D substructures may comprise before step (c) a further step comprising restriction of constructed chemical groups.

Restriction of constructed chemical groups may be achieved with an additional step (f) comprising selection of chemical groups at locations where the local atomic density is below a definite threshold.

In another particular aspect, the restriction may be achieved with an additional step (g) wherein the restriction of constructed chemical groups is achieved with a selection among:
automatic selection of most exposed chemical groups using a local density function and a threshold,
semi-automatic selection of chemical groups that are interacting with a given set of chemical groups, and
manual selection of subsets of chemical groups.

The process to identify similar 3D substructures may be performed by applying a refinement step comprising:
i) converting the pairs of clusters identified in step (d) to pairs of chemical groups, and
ii) minimizing the reciprocal distances between converted pairs of chemical groups with a distance function.

Eventually, steps (i) and (ii) are iteratively repeated using variable selection thresholds.

In particular examples, when the refining step is operated, reciprocal distances between converted pairs of chemical groups may be calculated by the function:
dist(g₁,g₂)=α·∥pos(g₁)−pos(g₂)∥+β·|D(g₁)−D(g₂)|+γ·orient(g₁,g₂)
wherein pos(g) is the position of the chemical group g after optimal superimposition of the given set of converted pairs, D(g) its local density, orient(g₁,g₂) is the difference of orientation between chemical groups g₁ and g₂ after optimal superimposition, and α, β and γ are normalising coefficients that are calculated such that the average value of each term is ⅓, on the basis of a statistical set of pairs of similar chemical groups.

The process to identify 3D similar substructures may further comprise a step (e) of clustering pairs of clusters identified in step (d) into a larger pair of clusters sharing similar 3D structures.

The 3D atomic structure having a plurality of individual atoms is a covalent or weak assembly of at least one molecule selected from the group comprising: natural and artificial proteins, oligopeptides, polypeptides, nucleic acids, natural and artificial oligonucleotides, natural and artificial oligosaccharides and polysaccharides, glycoproteins, lipoproteins, lipids, ions, water, natural and synthetic polymers, non polymeric structures, natural and artificial inorganic molecules.

In particular aspects, the 3D atomic substructure to be identified onto 3D atomic structures having a plurality of individual atoms is a functional site.

The functional site may be selected among the group comprising: enzymatic active sites, sites of reversible or irreversible binding of specific classes of molecules, sites sensitive to physico-chemical changes in the environment, chemical groups involved in energy conversion, self modification locations, antigenic parts of a molecule, mimetic sites, consensus sites, highly variable sites, sites necessary for initiating or interrupting a biological pathway, sites with particular physico-chemical properties, sites with particular chemical composition, protein taxons, immunoglobulin domains, DNA consensus sequences, gene expression signals, promoter elements, RNA processing signals, translational initiation sites, recognition motifs of a large variety of sequence-specific DNA-binding proteins, protein and nucleic acid compositional domains, glutamine-rich activation domains, CpG island, interaction site between a protein and a ligand, functional sugar binding site.

Also, the 3D atomic substructures to be identified onto 3D atomic structures having a plurality of individual atoms may be 3D structural sites issued from combinatorial, or conventional screening.

The invention also relates to a process to predict functional sites onto 3D atomic structures comprising the identification of 3D atomic substructures with a process further comprising correlation of the identified 3D atomic substructures with a known biological or chemical function.

Another aspect of the invention is a process to identify similar 3D atomic substructures A and B further comprising calculation of average orientation of the 3D atomic substructures A and B with respect to the orientation of their individual atoms and a visual representation of the A and B 3D atomic substructures, wherein the visual representation is operated by graphic projections matching the following conditions:
the average orientation of the 3D A atomic structure is orthogonal to the projection plane; and
the substructure B is optimally superimposed to the substructure A.

### EXAMPLE 1

Structural Similarities Among Serine Proteases

Subtilisin and γ-chymotrypsin are endoproteases sharing a similar catalytic site: both mechanisms use catalytic triad formed by an aspartate, a histidine and a serine. These proteins do not share either sequence similarity or similar fold in spite of their highly similar active sites. FIG. 8 shows that the position of these residues has neither the same position nor the same order within the sequence, making it irrelevant to align their sequences. Structures 1 SBC of subtilisin and 1AFQ of γ-chymotrypsin have been compared in accordance with aspects of the invention. The resulting file is shown in FIG. 9 and displays one similar region that consists of the catalytic triad (Asp32/Asp102, His64/His57, Ser221/Ser195) represented but four chemical groups, plus a glycine (Gly127/Gly216) which is also known to play a role in protease activity [18].

### EXAMPLE 2

Structural Similarities Between Legume Lectins

The structural family of legume lectins is represented by 106 structures publicly available in the PDB.

Many of them are functional lectins, i.e. proteins that bind oligosaccharides non-covalently, but some of them have lost the capability to bind sugar at this site in spite of their overall sequential and structural similarity (see [19] for a full review on lectins). Proteins without native sugar-binding ability are arcelin and α-amylases inhibitors (4 structures). Seven structures are available of demetallized lectins, i.e. lectins whose site has been deprived of Ca 2+ and Zn 2+. For example, 1DQ1 and 1DQ2 are 2 structures of concanavalin A in both native and demetallized forms: though their sequences are identical and their backbone have an RMSD of 0.9 Å for α-carbons, only the first form binds a sugar 3D atomic structure.

Structure 2PEL of the peanut lectin has been used to represent a functional lectin: its site of interaction with lactose has been selected and compared to every structure within the family. More precisely, all groups that have at least one atom closer than 4 Å to any atom of the ligand were selected. Thus, 10 chemical groups covering 9 aminoacids were retained (FIG. 11). The result of these comparisons is summarized in FIG. 10. Among all structures, 91 proteins showed at least one similar patch. All of these patches were sugar binding sites from functional proteins. No patch was detected among the 11 proteins missing the sugar-binding function. Thus, only 4 functional sites were not detected, and no false positive was obtained. Local conformational changes at the binding site explain the lost of activity in the case of demetallized lectins as shown on FIG. 12.

The legume lectins example shows that the process to identify 3D substructures excludes non-functional lectins by comparing them to a functional sugar binding site in spite of a high degree of similarity in sequence and in main chain architecture. This indicates that something like structural flexibility is taken into account by the process. It is sensitive enough to detect local conformation changes that are correlated with a loss of function. The process to identify 3D substructures is also flexible enough to ignore minor changes like those depending on the presence or absence of the ligand. Only 4% of the functional lectins were not detected.

The capabilities of this tool are illustrated and validated across two extreme kinds of biological problems: 1) the case of convergent evolution, in which proteins do not share any sequential or fold similarities, but share a common biochemical activity, is illustrated by two unrelated serine proteases; (2) the case of divergent evolution and loss of function due to minor modifications in the protein structure is illustrated by the analysis of the legume lectins family.

Selected advantages of this process reside in the fact that it:
(1) considers in a single process relevant information provided by the protein structure regarding protein function in its broadest sense;
(2) uses representations and strategies that fit the intuitive models for chemical interactions;
(3) chooses algorithmic strategies that allow for the search of the whole PDB for a site in less than one day; and
(4) makes the software customizable at run time.

The process of comparing 3D atomic structures allows for the detection of 3D structural similarities in 3D atomic structures. The 3D structural similarities are correctly detected in serine proteases that are differently folded and have unrelated sequences: Dali [20] finds no similarity between these structures and it is not possible to propose a valid sequence alignment due to the inversion of catalytic residues in the sequence.

In this case, the structural similarity that is automatically detected by the process of this invention corresponds to a common biochemical function and an identical catalytic mechanism.

In other cases, the invention compares structures of proteins which are known to act as competitors in a biological process that is understood with more or less precision: enzymatic catalysis, affinity for a ligand, disruption or activation of biochemical pathways, immunological cross-reactivity, inhibition of cell adhesion, etc.

The use of chemical groups to represent elementary bricks instead of amino acids to understand molecular functions is essential, but not possible if only the sequence of the protein is known.

Amino acids are composed of several critical groups that may or may not be important depending on the structural context (Table 1). Knowing the 3D structure of proteins allows one to model proteins with chemical groups, covalent bonds and other interactions independently from the concept of amino acid.

The choice of using triplets of chemical groups as the basic information for the comparison has been made for several reasons:
(1) a triangle may be associated with a number of parameters that ensures that a given triangle contains an amount of information that makes it much more rare than a single group represented by a point. It stands for a minimal representation of a local environment, including an oriented plan;
(2) a specific biological function is rarely provided by only one or two chemical groups;
(3) the basic information that consists of the chemical group type and its position is kept along all comparison steps; and
(4) adjacent triangles are easy to cluster to represent larger regions of 3D atomic structures.

A limit to the representation of a 3D atomic structure by a single graph of 3D located objects (such as points or triangles) is the difficulty to mix well-located and numerous objects (such as hydrogen bonds) with less located but sparser objects (such as clusters of 3 positively charged aminoacids).

The burial of atoms is not estimated using an accessible surface area (ASA) calculation, but a notion of local atomic density. In analogy to immerged bodies, the ASA would correspond to the emerged part of a floating body and be null for any object under the surface, whereas the density calculation is a measure of the depth of any object, even non-floating ones. FIG. 12 shows that aspartate in the catalytic triad of serine proteases is almost completely buried, suggesting that crucial residues may be essential for protein function, even if they lie below the surface of the 3D atomic structure. This kind of depth estimation is also essential for providing a vector that is roughly orthogonal to the molecular surface: these vectors are used to estimate the angle formed between a given triplet of chemical groups and the surface.

As the process may be used to perform a large number of comparisons, especially when one of the compared elements is a small site, the following useful strategies may be used:
compare two protein structures that have a poorly understood functional analogy;
screen the PDB for a given site; and
screen a database of functional 3D sites with a newly determined structure.

This process is essentially relevant to annotate newly determined structures from structural genomics approaches. It would require to build a valid database of sites. Even this work can be partially automated by considering as a site any set of chemical groups that interact with a ligand in the PDB.

### REFERENCES

The subject matter of the below listed references is incorporated herein by reference:
1. Altschul, S. F., et al., Gapped BLAST and PSI-BLAST: a new generation of protein database search programs. Nucleic Acids Res, 1997. 25(17): p. 3389-402.
2. Pearson, W. R., Rapid and sensitive sequence comparison with FASTP and FASTA. Methods Enzymol, 1990. 183: p. 63-98.
3. Bairoch, A., PROSITE: a dictionary of sites and patterns in proteins. Nucleic Acids Res, 1991. 19 Suppl: p. 2241-5.
4. Gribskov, M., A. D. McLachlan, and D. Eisenberg, Profile analysis: detection of distantly related proteins. Proc Natl Acad Sci USA, 1987. 84(13): p. 4355-8.
5. Blanchet, C., Logiciel MPSA et ressources bioinformatiques client-serveur Web dédiés á l'analyse de sequences de protéine. 1999, Université Claude Bernard, Lyon 1: Lyon, France.
6. Thompson, J. D., D. G. Higgins, and T. J. Gibson, CLUSTAL W: improving the sensitivity of progressive multiple sequence alignment through sequence weighting, position-specific gap penalties and weight matrix choice. Nucleic Acids Res, 1994. 22(22): p. 4673-80.
7. Holm, L. and C. Sander, Protein structure comparison by alignment of distance matrices. J. Mol. Biol., 1993. 233: p. 123-138.
8. Holm, L. and C. Sander, Touring protein fold space with Dali/FSSP. Nucleic Acids Res, 1998. 26(1): p. 316-9.
9. Fischer, D., et al., An efficient automated computer vision based technique for detection of three dimensional structural motifs in proteins. J Biomol Struct Dyn, 1992. 9(4): p. 769-89.
10. Lin, S. L., et al., Molecular surface representations by sparse critical points. Proteins, 1994. 18(1): p. 94-101.
11. Sandak, B., R. Nussinov, and H. J. Wolfson, An automated computer vision and robotics-based technique for 3-D flexible biomolecular docking and matching. Comput Appl Biosci, 1995. 11(1): p. 87-99.
12. Preissner, R., A. Goede, and C. Frommel, Dictionary of interfaces in proteins (DIP). Data bank of complementary molecular surface patches. J Mol Biol, 1998. 280(3): p. 535-50.
13. Preissner, R., A. Goede, and C. Frommel, Homonyms and synonyms in the Dictionary of Interfaces in Proteins (DIP). Bioinformatics, 1999. 15(10): p. 832-6.
14. Katchalski-Katzir, E., et al., Molecular surface recognition: determination of geometric fit between proteins and their ligands by correlation techniques. Proc Natl Acad Sci USA, 1992. 89(6): p. 2195-9.
15. Karlin, S. and Z. Y. Zhu, Characterizations of diverse residue clusters in protein three-dimensional structures. Proc Natl Acad Sci USA, 1996. 93(16): p. 8344-9.
16. Wei, L. and R. B. Altman, Recognizing protein binding sites using statistical descriptions of their 3D environments. Pac Symp Biocomput, 1998: p. 497-508.
17. Taroni, C., S. Jones, and J. M. Thornton, Analysis and prediction of carbohydrate binding sites. Protein Eng, 2000. 13(2): p. 89-98.
18. Voet, D. and J. Voet, Biochemistry. 1997, Wiley and Sons. p. 389-400.
19. Lis, H. and N. Sharon, Lectins: carbohydrate-specific proteins that mediate cellular recognition. Chem. Rev., 1998. 98: p. 637-674.
20. Holm, L. and C. Sander, Dali/FSSP classification of three-dimensional protein folds. Nucleic Acids Res, 1997. 25(1): p. 231-4.

1) A process for identifying similar 3D substructures onto 3D atomic structures having a plurality of individual atoms comprising:
a) attributing to each individual atom of the 3D atomic structure a structural parameter combining atomic local density D, local center of mass C and orientation in relation with position P;
b) constructing chemical groups by setting individual atoms having similar structural parameters;
c) constructing clusters of at least three chemical groups by setting the chemical groups whose reciprocal distances are constrained; and
d) comparing clusters constructed in step (c) and identifying the clusters sharing similar 3D structures.

2) The process according to claim 1, wherein the atomic local density D is calculated for each atom A on basis to position P defined by coordinates (xₚ, yₚ, zₚ) as a function of density m, modulated by a weight function w, according to:
$$D ( {x}_{P} , {y}_{P} , {z}_{P} ) = \frac{\begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot ⅆ x \cdot ⅆ y \cdot ⅆ z\end{matrix}}{{\int }_{- ∞}^{+ ∞} {\int }_{- ∞}^{+ ∞} {\int }_{- ∞}^{+ ∞} w ( x , y , z ) \cdot ⅆ x \cdot ⅆ y \cdot ⅆ z}$$

3) The process according to claim 2, wherein the weight function w is a spherical function
$$w ( x , y , z ) = \frac{1}{4} ( 1 - \frac{r}{{r}_{c}} )$$
if r≦r, 0 otherwise
where r is \sqrt{x²+y²+z²}, r_{c} a critical radius and the factor [¼] allows to make r independent from r_{c} if m is constant.

4) The process according to claim 1, wherein a local center of mass C(P) for a given atom A occupying a position P is calculated as a point which cartesian coordinates (x_{c}, y_{c}, z_{c}) match the following:
$$\{ \begin{matrix}{x}_{C} ( {x}_{P} , {y}_{P} , {z}_{P} ) = \frac{ \begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot x \cdot ⅆ x \cdot ⅆ y \cdot ⅆ z\end{matrix}}{\begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot ⅆ x \cdot ⅆ y \cdot ⅆ z\end{matrix}} \\ {y}_{C} ( {x}_{P} , {y}_{P} , {z}_{P} ) = \frac{ \begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot y \cdot ⅆ x \cdot ⅆ y \cdot ⅆ z\end{matrix}}{\begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot ⅆ x \cdot ⅆ y \cdot ⅆ z\end{matrix}} \\ {z}_{C} ( {x}_{P} , {y}_{P} , {z}_{P} ) = \frac{ \begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot z \cdot ⅆ x \cdot ⅆ y \cdot ⅆ z\end{matrix}}{\begin{matrix}{\int }_{- ∞}^{+ ∞} {\int }_{- ∞}^{+ ∞} {\int }_{- ∞}^{+ ∞} w ( x - {x}_{P} , y - {y}_{P} , z - {z}_{P} ) \cdot \\ m ( x , y , z ) \cdot ⅆ x \cdot ⅆ y \cdot ⅆ z\end{matrix}}\end{matrix}$$
where x, y and z are spatial coordinates, m is the density function and w is a weight function.

5) The process according to claim 4, wherein the weight function w is a spherical function:
$$w ( x , y , z ) = \frac{1}{4} ( 1 - \frac{r}{{r}_{c}} )$$
if r≦r_{c}, 0 otherwise
where r is \sqrt{x²+y²+z²}, r_{c} a critical radius and the factor [¼] allows to make r independent from r_{c}, if m is constant.

6) The process according to claim 1, wherein in step (a), orientation of each atom A occupying a position P and having a local center of mass C(P) is calculated by a density gradient represented by a vector \vec{CP}.

7) The process according to claim 1, wherein reciprocal distances between chemical groups in step (c) are 2 to 20 Å.

8) The process according to claim 1, wherein constructing clusters in step (c) comprises orientation of the clusters against the 3D atomic structure.

9) The process according to claim 8, wherein orientation of the constructed cluster against the 3D atomic structure is operated by a scalar triple product of three vectors \vec{CP}ᵢ, \vec{CP}ⱼ, \vec{CP}ₖ, wherein C is the center of the local centers of mass of each chemical group and Pᵢ, Pⱼ, Pₖ are three distinct points in the cluster.

10) The process according to claim 1, wherein clusters of chemical groups are further stored in a database.

11) The process according to claim 1, wherein comparison of a given pair of clusters in step (d) comprises identification of at least one structural similarity selected from the group consisting of:
same chemical groups and similar orientation,
similar length of reciprocal distances between chemical groups,
similar local density of the chemical groups,
similar orientation of the constructed clusters, and
capability of binding flexible ligands using the same kind of weak chemical bonds.

12) The process according to claim 11, wherein after identification of at least two structural similarities, a global score is calculated as a function combining several parameters indicating similarity of the clusters, the parameters being selected from the group consisting of:
volume of chemical groups,
scarcity of the chemical groups,
quality of the superposition with respect to the standard deviation,
quality of the superposition with respect to the orientation of the chemical groups, and
resemblance of the atomic environment.

13) The process according to claim 12, applied to the capability of binding flexible ligands using the same kind of weak chemical bonds.

14) The process according to claim 13, wherein the capability may be estimated by analysis of deviation of short range distances between chemical groups.

15) The process according to claim 12, wherein the resemblance between atomic environment comprises calculation of a score by comparing volumes of atoms around each converted pair of chemical groups.

16) The process according to claim 15, wherein the score is calculated by the function:
$$s ( {v}_{1} , {v}_{2} , v ) = 1 - \frac{2 v - ( {v}_{1} + {v}_{2} )}{v}$$
wherein:
V₁ is the volume of atoms surrounding chemical groups in cluster (3D atomic substructure) extracted from atomic structure M₁;
V₂ is the volume of atoms surrounding chemical groups in cluster (3D atomic substructure) extracted from atomic structure M₂; and
V is the volume of atoms surrounding chemical groups in both clusters after superposition of the the clusters.

17) The process according to claim 1, wherein before step (c) a further step (f) comprising the restriction of constructed chemical groups is performed by selection of chemical groups at locations where the local atomic density is below a definite threshold.

18. The process according to claim 1, wherein before step (c), a further step (g) comprising restriction of constructed chemical groups is performed from at least one step of:
automatic selection of most exposed chemical groups using a local density function and a selected threshold,
semi-automatic selection of chemical groups that are interacting with a given set of chemical groups, and
manual selection of subsets of chemical groups.

19) The process according to claim 1, further comprising a refinement step comprising:
i) converting the pairs of clusters identified in step (d) to pairs of chemical groups, and
ii) minimizing reciprocal distances between converted pairs of chemical groups by a distance function.

20) The process according to claim 19, wherein steps (i) and (ii) are iteratively repeated using variable selection thresholds.

21) The process according to claim 19, wherein the reciprocal distances between converted pairs of chemical groups are calculated by the function:
dist(g₁,g₂)=α·∥pos(g₁)−pos(g₂)∥+β·|D(g₁)−D(g₂)|+γ·orient(g₁,g₂)
wherein pos(g) is the position of chemical group g after optimal superimposition of a given set of converted pairs, D(g) local density, orient(g₁,g₂) is the difference of orientation between chemical groups g₁ and g₂ after optimal superimposition, and α, β and γ are normalizing coefficients calculated such that the average value of each term is ⅓, on the basis of a statistical set of pairs of similar chemical groups.

22) The process according to claim 1, further comprising a step (e) of clustering pairs of clusters identified in step (d) into a larger pair of clusters sharing similar 3D structures.

23) The process according to claim 1, wherein the 3D atomic structure having a plurality of individual atoms is a covalent or weak assembly of at least one molecule selected from the group consisting of natural and artificial proteins, oligopeptides, polypeptides, nucleic acids, natural and artificial oligonucleotides, natural and artificial oligosaccharides and polysaccharides, glycoproteins, lipoproteins, lipids, ions, water, natural and synthetic polymers, non polymeric structures, and natural and artificial inorganic molecules.

24) The process according to claim 1, wherein the 3D atomic substructure is a functional site.

25) The process according to claim 24, wherein the functional site is selected from the group consisting of enzymatic active sites, sites of reversible or irreversible binding of specific classes of molecules, sites sensitive to physico-chemical changes in the environment, chemical groups involved in energy conversion, self modification locations, antigenic parts of a molecule, mimetic sites, consensus sites, highly variable sites, sites necessary for initiating or interrupting a biological pathway, sites with particular physico-chemical properties, sites with particular chemical composition, protein taxons, immunoglobulin domains, DNA consensus sequences, gene expression signals, promoter elements, RNA processing signals, translational initiation sites, recognition motifs of a large variety of sequence-specific DNA-binding proteins, protein and nucleic acid compositional domains, glutamine-rich activation domains, CpG island, interaction site between a protein and a ligand, and functional sugar binding site.

26) The process according to claim 1, wherein the 3D atomic substructures are 3D structural sites obtained from combinatorial, or conventional screening.

27) A process to predict functional sites onto 3D atomic structures comprising identifying 3D atomic substructures by a process according to claim 1, comprising correlation of the identified 3D atomic substructures with a known biological or chemical function.

28) The process according to claim 1, further comprising calculation of average orientation of 3D atomic substructures A and B with respect to orientation of their individual atoms and a visual representation of A and B 3D atomic substructures, wherein the visual representation is operated by graphs projections matching the following conditions:
a) the average orientation of the 3D A atomic structure is orthogonal to the projection plane; and
b) thee substructure B is optimally superimposed on the substructure A.
